(19) United States
(12) Patent Application Publication
(10) Pub. No.: US 2007/0248206 A1
(43) Pub. Date: Oct. 25, 2007

(54) CT SCANNER WITH UNTRACKED MARKERS

(76) Inventors: Predrag Sukovic, Birmingham, MI (US); William C. Van Kampen, Saline, MI (US); Joseph Webster Stayman, Ann Arbor, MI (US); Miodrag Rakic, Redondo Beach, CA (US); James A. Bertolina, Portage, MI (US); David Phillipe Sarment, Ann Arbor, MI (US); James F. O'Connell, Ann Arbor, MI (US)

Correspondence Address: CARLSON, GASKEY & OLDS, P.C., 400 WEST MAPLE ROAD, SUITE 350, BIRMINGHAM, MI, 48009, US

(21) Appl. No.: 11/737,238

(22) Filed: Apr. 19, 2007

Related U.S. Application Data
(60) Provisional application No. 60/793,153, filed on Apr. 19, 2006.
(60) Provisional application No. 60/851,196, filed on Oct. 12, 2006.

(51) Int. Cl. H05G 1/60; A61B 6/00; G01N 23/00; G21K 1/12

(52) U.S. Cl. 378/4

(57) ABSTRACT

A surgeon selects a volume of interest by placing an untracked “marker” in a patient near an area where an update is desired. During surgery, when an updated CT scan is requested, the CT scanner performs a scan of the patient using a full field of view to take a series of two-dimensional initial images of the patient from a plurality of angularly spaced positions about the patient. The position of the untracked marker is determined by the CT scanner in or more of the initial images. The volume of interest is defined as the position of the untracked marker, plus some margin. The CT scanner then collimates the x-ray source to scan only the volume of interest. The CT scanner then completes the update scan of the volume of interest and updates a previous CT scan(s) to create a fully updated CT image, reducing x-ray exposure of the patient.

### REFERENCE TO RELATED APPLICATIONS

This application claims priority to U.S. Provisional Application Nos. 60/793,153 filed Apr. 19, 2006 and 60/851,196 filed Oct. 12, 2006.

### BACKGROUND OF THE INVENTION

The present invention relates generally to a surgical imaging system including a CT scanner that scans a volume of interest of a patient as marked with an untracked marker and updates previous data with data from the volume of interest scan to create a fully updated CT image.

It is sometimes desirable to be able to take a CT scan of a patient during surgery. For example, a surgeon may want to check the progress of the surgery (e.g., to determine whether a problem has been completely corrected or whether a tumor has been completely removed, etc).

For image-guided surgery, it is sometimes desirable to periodically update a pre-operative CT scan of the patient. The relevant volume of the patient may have shifted between the time the pre-operative image was taken and the time of surgery. This is especially true once surgery has begun. For example, in cranial surgery, a shape of an intracranial cavity changes as the surgeon gains access. Changes in the pre-operative image and the actual surgical subject introduce variations into the surgical process. In matters like intracranial surgery, the tolerance for variations is low, thus even small changes between the image and actual subject may cause problems and make the surgery less effective.

To solve this problem, a new, partial CT scan may be taken during surgery to update the previously received information. It is known that a baseline, pre-operative CT scan can be updated with a partial CT scan of a volume of interest in which an x-ray source is collimated to scan only the volume of interest. The partial CT scan is used in conjunction with the pre-operative CT scan (which includes volumes that have presumably not changed) to obtain a full CT image.

However, finding the volume of interest on the pre-operative CT scan can be time-consuming. Additionally, if the patient and/or the CT scanner have moved, the relative locations of the CT scanner and patient must be determined and registered with the image guided surgical system before the location of the volume of interest can be determined.

### SUMMARY OF THE INVENTION

A surgeon selects a volume of interest in a patient by placing an untracked “marker” near an area where an updated is desired. The volume of interest is defined at the position of the untracked marker, plus some margin. The untracked marker is not tracked by a navigation system, and a position of the untracked marker is not determined by any additional hardware. The untracked marker is simply detectable in CT images. For example, the untracked marker can be radio-opaque or radio-translucent.

The position of the untracked marker is determined by a CT scanner in one or more (but significantly less than a full set) of frames. During surgery, when an updated CT scan is requested, the CT scanner begins performing a scan of a patient using a full field of view. The CT scanner takes a series of two-dimensional initial images (or “frames”) of the patient from a plurality of angularly spaced positions about the patient. The CT scanner performs two functions with the initial images. When a sufficient number of initial images have been obtained, the CT scanner determines the location of the untracked marker relative to the CT scanner. The CT scanner also registers its location relative to the patient and the previous CT images based upon the initial images. In other words, based on the initial images, the CT scanner determines its position relative to the current position of the patient and the current position of the patient relative to the previous CT scan(s).

The CT scanner then collimates the x-ray source to scan only the volume of interest. The CT scanner then completes the update scan of the volume of interest, thereby updating the previous CT scan(s) while reducing x-ray exposure of the patient. The collimated scan could be done at a higher resolution than the previous CT scan(s) or the initial images.

### BRIEF DESCRIPTION OF THE DRAWINGS

FIG. 1 schematically illustrates a first embodiment CT scanner;

FIG. 2 illustrates the CT scanner of FIG. 1 with a part of a patient received in the CT scanner;

FIG. 3 illustrates a second embodiment of the CT scanner;

FIG. 4 illustrates a computer employed with the CT scanner;

FIG. 5 illustrates a first full field of view of a two-dimensional CT image;

FIG. 6 illustrates a second full field of view of a two-dimensional CT image; and

FIG. 7 illustrates a collimated field of view of a two-dimensional CT image that focuses on a volume of interest;

FIG. 8 illustrates an untracked marker including an image;

FIG. 9 illustrates a top view of a mouth guard including untracked markers; and

FIG. 10 illustrates a side view of the mouth guard including untracked markers that is placed between teeth of the patient.

### DETAILED DESCRIPTION OF THE PREFERRED EMBODIMENT

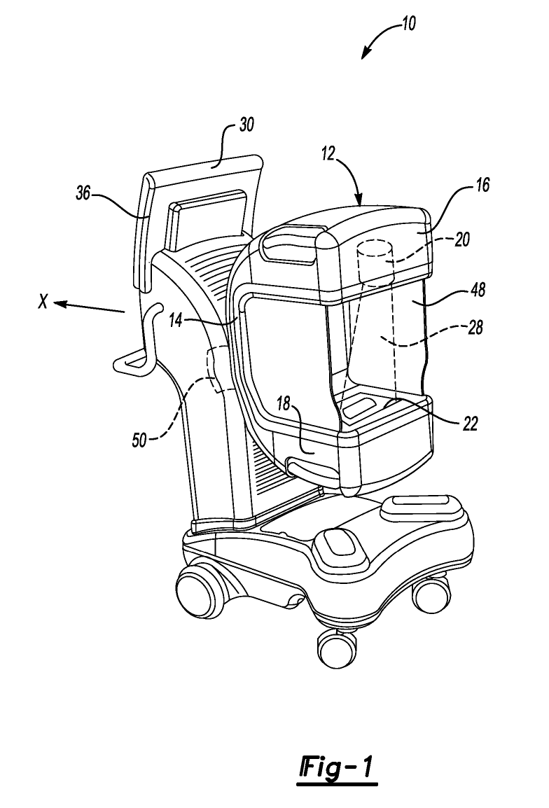

FIG. 1 illustrates an intra-operative CT scanner 10 of a surgical imaging system of the present invention including a gantry 12 that supports and houses components of the CT scanner 10. Suitable CT scanners 10 are known. In one example, the gantry 12 includes a cross-bar section 14, and a first arm 16 and a second arm 18 each extend substantially perpendicularly from opposing ends of the cross-bar section 14 to form the c-shaped gantry 12. The first arm 16 houses an x-ray source 20 that generate x-rays 28. In one example, the x-ray source 20 is a cone-beam x-ray source. The second arm 18 houses a complementary flat-panel detector 22 spaced apart from the x-ray source 20. The x-rays 28 are directed toward the detector 22 which includes a converter (not shown) that converts the x-rays 28 from the x-ray source 20 to visible light and an array of photodetectors behind the converter to create an image. As the gantry 12 rotates about the patient P, the detector 22 takes a plurality of x-ray images at a plurality of rotational positions. Various configurations and types of x-ray sources 20 and detectors 22 can be utilized, and the invention is largely independent of the specific technology used for the CT scanner 10.

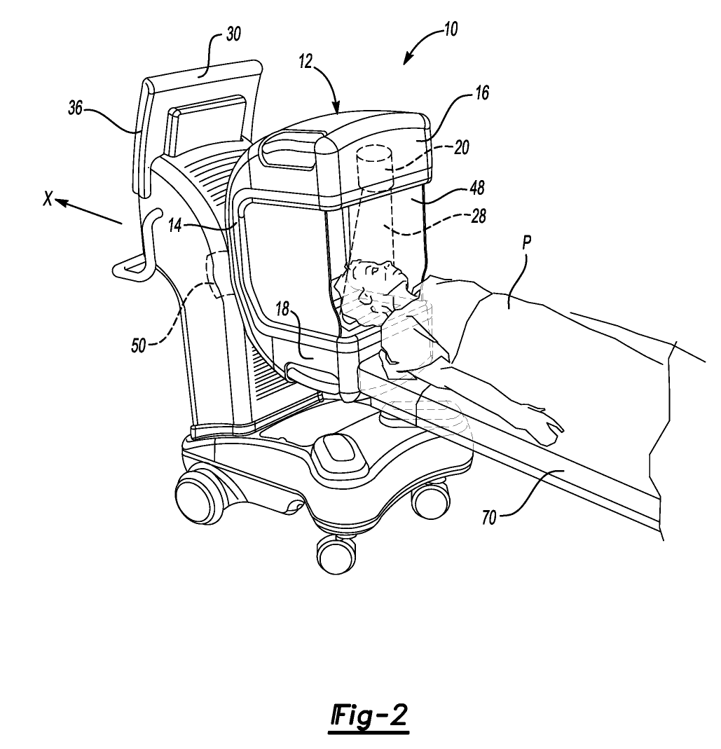

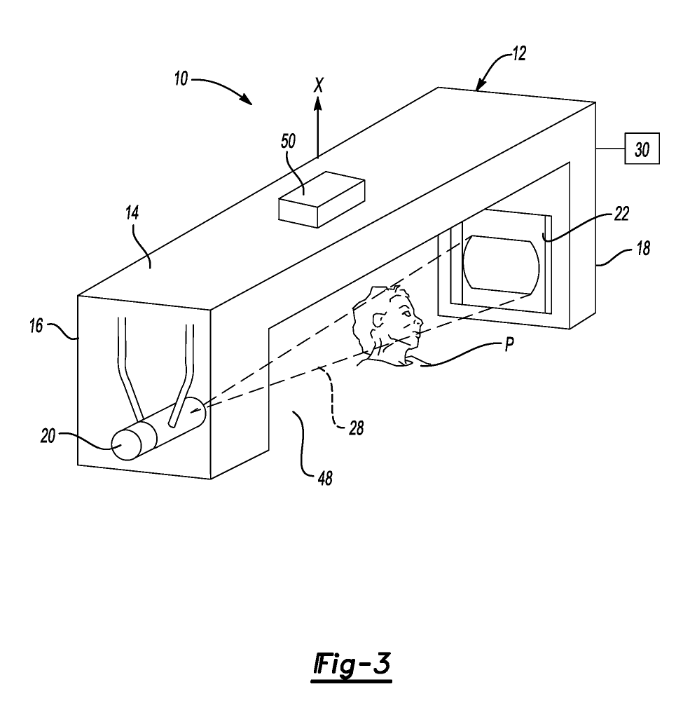

FIG. 2 illustrates the CT scanner 10 with a part of the patient P received in a space 48 between the first arm 16 and the second arm 18. A motor 50 rotates the gantry 12 about an axis of rotation X to obtain a plurality of x-ray images of the patient P at the plurality of rotational positions. The axis of rotation X is substantially centered within the gantry 12 and positioned between the x-ray source 20 and the detector 22. The gantry 12 can be rotated approximately slightly more than 360 degrees about the axis of rotation X. In one example, as shown in FIGS. 1 and 2, the axis of rotation X is substantially horizontal. In this example, the patient P is typically lying down on a table 70. Alternatively, as shown in FIG. 3, the axis of rotation X is substantially vertical. Typically, in this example, the patient P is sitting upright.

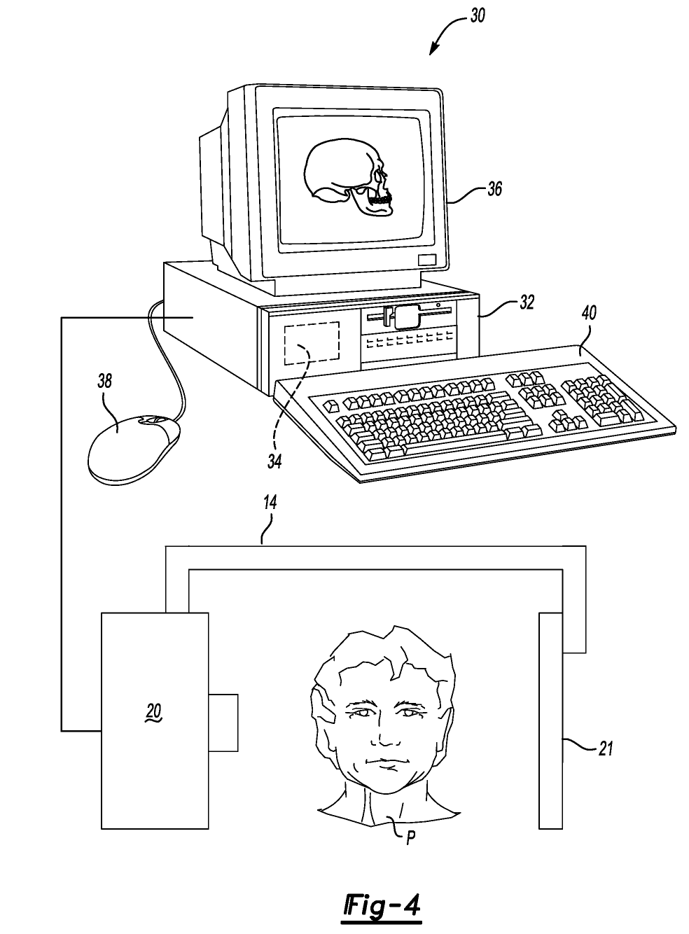

As shown schematically in FIG. 4, the CT scanner 10 further includes a computer 30 having a microprocessor or CPU 32, a storage 34 (memory, hard drive, optical, and/or magnetic, etc), a display 36, a mouse 38, a keyboard 40 and other hardware and software for performing the functions described herein. The computer 30 powers and controls the x-ray source 20 and the motor 50. The plurality of x-ray images taken by the detector 22 are sent to the computer 30. The computer 30 generates a three-dimensional CT image from the plurality of x-ray images utilizing any known techniques and algorithms. The three-dimensional CT image is stored on the storage 34 of the computer 30 and can be displayed on the display 36 for viewing.

Returning to FIG. 1, prior to surgery, a full pre-operative scan (CT or MRI) of the patient P is performed and stored on the computer 30. As an alternative, for some types of surgery, it is possible that some generic data (i.e., data not specifically from the present patient P) describing the area surrounding a volume of interest 59 (described below) may be sufficient. The pre-operative data may be a complete three-dimensional CT image or model or a partial three-dimensional CT image or model of the area surrounding the volume of interest 59. Alternately, data can be generated from an intra-operative scan instead of using pre-operative data.

During surgery, the CT scanner 10 takes intra-operative CT scans of the volume of interest 59 within the patient P so that the surgeon (or a dentist) can determine the current progress of the surgery (e.g., has a tumor been completely removed or a sinus cavity been completely repaired?) The CT scanner 10 only performs a complete CT scan of the volume of interest 59, which is the volume (or volumes) where the surgeon is working.

The computer 30 uses the pre-operative data surrounding the volume of interest 59 in conjunction with the new information from the intra-operative CT scans of the volume of interest 59 to create a fully updated three-dimensional CT image. Therefore, a new, full intra-operative CT scan is not required to form a CT image of the volume of interest 59. The smaller scan of the volume of interest 59 also reduces the dosage of x-rays experienced by the patient P.

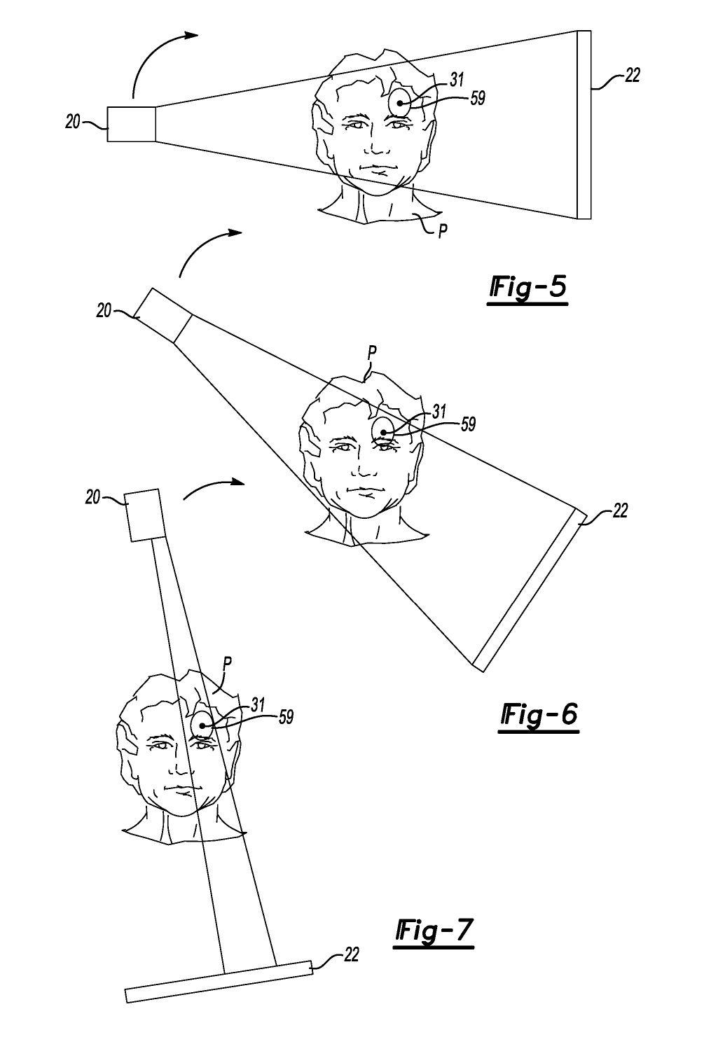

When a surgeon determines that an updated CT image is needed, the surgeon can request a manually designated updated CT scan of the volume of interest 59. The surgeon places an untracked marker 31 in the patient P near the area where an update is desired, as shown in FIG. 5. The untracked marker 31 may be a simple spherical object (like a ball bearing or bead, etc.) that is visible in the CT images. The untracked marker 31 is preferably substantially radio-opaque (i.e., opaque to x-rays), but may also be somewhat radio-translucent. For example, the untracked marker 31 can be a metal BB. A radio-transparent string, ribbon or strand may be connected to the untracked marker 31 to facilitate subsequent location and removal. The volume of interest 59 is defined as the location of the untracked marker 31, plus some margin.

When an updated CT scan is requested, the CT scanner 10 takes a series of full field of view, two-dimensional CT images (initial images) from a plurality of angularly separated positions about the patient P (as shown in FIGS. 5 and 6, although more images could be used). The plurality of positions may be the same angularly-spaced positions that would be used in a full CT scan or may be separated by much larger angles (so that fewer positions could be used). For an illustrative example only, between two and ten initial images could be taken over approximately 45 degrees.

The initial images provide two primary purposes: 1) to determine the position of the CT scanner 10 relative to the patient P (who may have been moved during the surgery); and 2) to determine the location of the untracked marker(s) 31 to define the volume(s) of interest 59. These two purposes may be accomplished in either sequence. Additionally, the initial images (or portions of them) may be used to perform the update.

Based upon the initial images, the CT scanner 10 registers its location relative to the patient P (who may have moved during surgery) and the previous CT scans. This can be done by locating and orienting a known structure in this part of the patient's P anatomy (e.g., part of the skull) that can be found in the previous CT scans. The CT scanner also (before, after or simultaneously) determines the location of each of the untracked markers 31 (if more than one). The feature of registering the location of the CT scanner 10 relative to the current patient P location could be done independently of the volume of interest 59 feature, and vice versa.

The CT scanner 10 then collimates the x-ray source 20 and takes a plurality of images at a plurality of angularly-spaced positions to perform the intra-operative updated CT scan of the volume of interest 59, as shown in FIG. 7. After collimating, the CT scanner 10 takes images at the regularly spaced intervals for the remainder of the CT scan (approximately 180 to 360 degrees).

The computer 30 uses the new information from the intra-operative updated CT scans of the volume of interest 59 in conjunction with the pre-operative data surrounding the volume of interest 59 to create a fully updated three-dimensional CT image.

If more than one untracked marker 31 is used, the CT scanner 10 could present the surgeon with the option of choosing one or more of the volumes of interest(s) 59. If more than one volume of interest 59 is selected, the CT scanner 10 could 1) collimate to obtain images for each of the volumes of interest 59 in alternating frames as the CT scanner 10 rotates around the patient P, 2) the CT scanner 10 could perform multiple rotations about the patient P (or multiple 180 degree scans, or anywhere between 180 and 360 degrees for each scan), or 3) define a single volume of interest 59 large enough to accommodate all of the untracked markers 31, plus some margin.

The CT scanner 10 then automatically (i.e., without further prompting or input) displays the volume of interest 59 on the display 36. If more than one volume of interest 59 was selected, the CT scanner 10 marks the locations of the volumes of interest 59 relative to the CT scan so the surgeon can easily toggle or scroll between the volumes of interest 59.

Alternately, the surgeon can request a manually designated updated CT scan of the volume of interest 59 by using a graphical user interface (or voice activated user interface) on the computer 30. After the CT scanner 10 locates and display a volume of change by comparing the initial images to the pre-operative data, the surgeon can manually select the volume of interest 59 on the previously-stored CT image using software on the computer 30. For example, the surgeon can circle the volume of interest 59 on a three dimensional CT image on the computer 30 by using the mouse 38 or the keyboard 40. The CT scanner 10 then determines its location relative to the patient P based upon the initial images. Then, with its location registered relative to the patient P, the CT scanner 10 focuses in on the volume of interest 59 and completes the updated CT scan.

Alternately, the surgeon can request an automatically designated updated CT scan of the volume of interest 59. The CT scanner 10 compares the initial images to the pre-operative data to determine a volume of change. After locating the volume of change, the CT scanner then defines the volume of interest 59 as the volume of change, plus some margin. The CT scanner 10 focuses in on the volume of interest 59 and completes the updated CT scan

Because the pre-operative data is used only for background information and calculations required in creating a new image, the pre-operative data has lesser importance in the new image than does the intra-operative data. As a result, the pre-operative scan may use a lower dosage and/or a lower resolution than would otherwise be used. For example, the CT scanner 10 begins scanning the patient P at a low resolution, full field of view to take the initial images until the position(s) of the untracked marker(s) 31 is determined. The x-ray source 20 is then collimated to the volume of interest 59 around the untracked marker 31, and a high resolution scan of the volume of interest 59 is performed. This results in a safer pre-operative scan for the patient P and a cost savings in obtaining the pre-operative scan.

When a high resolution updated CT scan of the volume of interest 59 is taken, the untracked marker 31 is shown on the display 36 in a three-dimensional CT image. As shown in FIG. 8, the untracked marker 31 can include an image 64 on or in the untracked marker 31. For example, the image 64 can be lines, text or any marking. The image 64 can be defined by contrasting radio-transparent and radio-opaque materials.

After the updated CT scan is taken, such as at a higher resolution, the image 64 on the untracked marker 31 should be visible on the display 36. If the image 64 is clearly visible on the untracked marker 31 in the updated CT scan, this indicates that the updated CT scan of the volume of interest 59 is a good scan. However, if the image 64 on the untracked marker 31 is not clearly visible on the display 36, this indicates that the updated CT scan of the volume of interest 59 is not a good scan, possible due to movement or other causes, and another updated CT scan of the volume of interest 59 is needed.

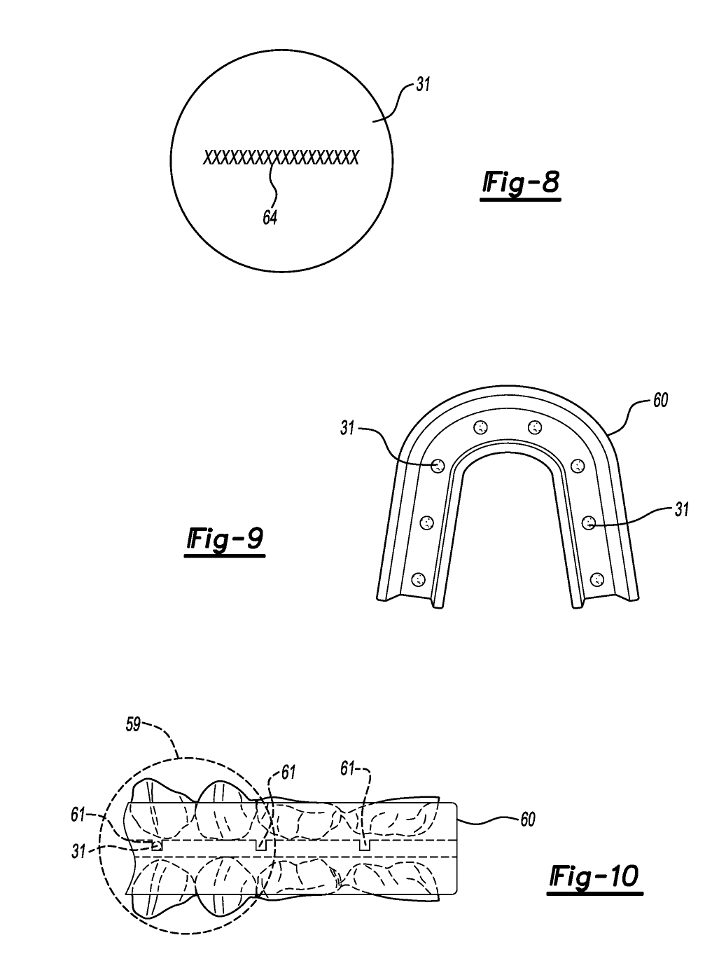

As shown in FIGS. 9 and 10, the untracked marker 31 can be used in a dental procedure to mark the volume of interest 59 and optionally detect movement of the patient P. In one example, the volume of interest 59 is one or two teeth. In one example, the untracked marker 31 is located in a radio-transparent polymer mouth appliance 60 (similar to a sports mouth guard) placed between the teeth T of the patient P. The mouth appliance 60 includes a plurality of pockets 61 formed therein. Although only one row is shown, other arrangements or arrays of pockets 61 could be implemented. At least one untracked marker 31 is selectively placed in the pockets 61 by the doctor or technician to indicate the volume of interest 59. Alternately, the untracked marker 31 can be air (i.e., the lack of an object in a pocket 61). As described above, a CT scan of the volume of interest 59 can be taken at high-resolution, and the surrounding areas can be scanned at a lower resolution.

The foregoing description is only exemplary of the principles of the invention. Many modifications and variations are possible in light of the above teachings. It is, therefore, to be understood that within the scope of the appended claims, the invention may be practiced otherwise than using the example embodiments which have been specifically described. For that reason the following claims should be studied to determine the true scope and content of this invention.

What is claimed is:

1. A method of updating a CT scan of a patient, the method comprising the steps of:
positioning a marker in a patient;
taking a plurality of x-ray images of the patient to obtain initial images;
using the initial images to determine a location of the marker;
defining a volume of interest based upon the location of the marker;
collimating an x-ray source based upon the volume of interest to direct x-rays towards the volume of interest;
performing a collimated intra-operative CT scan of the volume of interest to obtain collimated x-ray data; and
reconstructing a CT image based upon previous data and the collimated x-ray data to create a fully updated CT image.

2. The method as recited in claim 1 further including the step of obtaining the previous data by performing a pre-operative CT scan of the patient and storing the previous data on a computer.

3. The method as recited in claim 1 further including the step of obtaining the previous data from generic data.

4. The method as recited in claim 1 wherein the step of collimating the x-ray source is done with a collimated field of view, and the step of taking the plurality of x-ray images is done with a full field of view that is larger than the collimated field of view.

5. The method as recited in claim 1 wherein a number of the initial images taken during the step of taking the plurality of x-ray images is substantially less than a number of collimated x-ray images taken during the step of performing the collimated intra-operative CT scan.

6. The method as recited in claim 1 wherein the initial images are taken over an angular area of less than 45°

7. The method as recited in claim 1 wherein the marker is one of radio-opaque and radio-translucent.

8. The method as recited in claim 1 wherein the marker is spherical.

9. The method as recited in claim 1 further including the step of visually identifying an image on the marker in the fully updated CT image to confirm a proper scan.

10. The method as recited in claim 1 wherein the step of taking the plurality of x-ray images generates partial intra-operative data, and the collimated x-ray data has a higher resolution than the partial intra-operative data and the previous data.

11. A CT scanner comprising:
an x-ray source to generate x-rays;
an x-ray detector mounted opposite the x-ray source;
a marker located in a patient; and
a computer that stores previous data and uses initial images to determine a location of the marker in the patient and defines a volume of interest based upon the location of the marker, wherein the x-ray source is then collimated to focus collimated x-rays towards the volume of interest to obtain collimated x-ray data of the volume of interest, and the computer creates a CT image based upon the previous data and the collimated x-ray data to obtain a fully updated CT image.

12. The CT scanner as recited in claim 11 wherein the x-ray source is a cone-beam x-ray source.

13. The CT scanner as recited in claim 11 further including a gantry including a cross-bar section, a first arm and a second arm that each extend substantially perpendicularly to the cross-bar section, wherein the x-ray source is housed in the first arm and the x-ray detector is housed in the second arm.

14. The CT scanner as recited in claim 11 wherein the previous data is one of generic data and a pre-operative scan of the patient.

15. The CT scanner as recited in claim 11 wherein the previous data and x-ray data from the initial images have a lower resolution than the collimated x-ray data.

16. The CT scanner as recited in claim 11 wherein the marker is one of radio-opaque and radio-translucent.

17. The CT scanner as recited in claim 11 wherein the marker is spherical.

18. The CT scanner as recited in claim 1 wherein the marker includes an image, and the image is readable on the fully updated CT image to confirm a proper scan.

19. A method of updating a CT scan of a patient, the method comprising the steps of:
positioning a marker in a patient;
taking a plurality of x-ray images of the patient to obtain initial images at a first resolution;
using the initial images to determine a location of the marker;
defining a volume of interest based upon the location of the marker;
directing x-rays towards the volume of interest; and
performing a CT scan of the volume of interest to obtain x-ray data at a second resolution, wherein the first resolution is lower than the second resolution.

20. The method as recited in claim 19 further including the step of reconstructing a CT image based upon previous data and the x-ray data to create a fully updated CT image.

21. The method as recited in claim 20 further including the step of visually identifying an image on the marker in the fully updated CT image to confirm a proper scan.

22. The method as recited in claim 20 further including the step of obtaining the previous data including one of performing a pre-operative scan of the patient and obtaining the previous data from generic data.

23. The method as recited in claim 19 further including the step of downsampling data from the initial images.

24. The method as recited in claim 23 wherein the step of downsampling includes one of sampling pixels of the data from the initial images and averaging together a signal from adjacent pixels of the data from the initial images.

25. A method of updating a CT scan of a patient, the method comprising the steps of:
obtaining CT data at a first resolution;
positioning a marker in a patient;
defining a volume of interest based upon the location of the marker;
directing x-rays towards the volume of interest; and
performing a CT scan of the volume of interest to obtain x-ray data at a second resolution, wherein the first resolution is less than the second resolution.

26. The method as recited in claim 25 wherein the step of obtaining the CT data includes one of performing a pre-operative scan of the patient and obtaining the previous data from generic data.

27. The method as recited in claim 26 further including the step of reconstructing a CT image based upon the CT data and the x-ray data to create a fully updated CT image.

28. The method as recited in claim 27 further including the step of visually identifying an image on the marker in the fully updated CT image to confirm a proper scan.

29. The method as recited in claim 25 wherein the step of obtaining the CT data includes taking a plurality of x-ray images to obtain initial images, the method further including the step of using the initial images to determine the location of the marker.

30. The method as recited in claim 25 further including the step of downsampling the CT data.

31. The method as recited in claim 30 wherein the step of downsampling includes one of sampling pixels of the data from the initial images and averaging together a signal from adjacent pixels of the data from the initial images.

32. A method of confirming a proper CT scan, the method comprising the steps of:
positioning a marker in a patient; and
visually identifying an image on the marker in a CT image to confirm a proper scan.

33. The method as recited in claim 32 further including the steps of:
taking a plurality of x-ray images of the patient to obtain initial images,
using the initial images to determine a location of the marker,
defining a volume of interest based upon the location of the marker,
collimating an x-ray source based upon the volume of interest to direct x-rays towards the volume of interest,
performing a collimated intra-operative CT scan of the volume of interest to obtain collimated x-ray data, and
reconstructing the CT image based upon previous data and the collimated x-ray data to create a fully updated CT image.

34. The method as recited in claim 32 wherein the marker is one of radio-opaque and radio-translucent.

35. The method as recited in claim 32 wherein the marker is spherical.

36. A CT scanner comprising:
a marker located in a patient, the marker including an image; and
a computer that creates a CT image, wherein the image is visually identifiable in the CT image to confirm a proper scan.

37. The CT scanner as recited in claim 36 further including:
an x-ray source to generate x-rays,
an x-ray detector mounted opposite the x-ray source,
a computer that stores previous data and uses initial images to determine a location of the marker in the patient and defines a volume of interest based upon the location of the marker, wherein the x-ray source is then collimated to focus collimated x-rays towards the volume of interest to obtain collimated x-ray data of the volume of interest, and the computer creates the CT image based upon the previous data and the collimated x-ray data to obtain a fully updated CT image.

38. The method as recited in claim 37 wherein the marker is one of radio-opaque and radio-translucent.

39. The method as recited in claim 37 wherein the marker is spherical.
